(19) United States
(12) Patent Application Publication
(10) Pub. No.: US 2003/0199001 A1
(43) Pub. Date: Oct. 23, 2003

(54) Sample preparation of biological fluids for proteomic applications

(76) Inventors: Aldo M. Pitt, Wayland, MA (US); Sara D. Gutierrez, Danvers, MA (US)

Correspondence Address: MILLIPORE CORPORATION, 290 CONCORD ROAD, BILLERICA, MA, 01821, US

(21) Appl. No.: 10/419,374

(22) Filed: Apr. 21, 2003

Related U.S. Application Data
(60) Provisional application No. 60/375,199, filed on Apr. 23, 2002.

(51) Int. Cl. G01N 33/53; C12N 9/00; C07K 14/00; C07K 1/34

(52) U.S. Cl. 435/7.1; 435/183; 530/414

(57) ABSTRACT

A system, kit and process for the separation, recovery, purification and identification of biomarkers such as peptides and the like from serum or plasma. The process is to filter the sample through a UF filtration device optionally having a vertical or substantial vertical ultrafilter membrane, especially when in a single well configuration device. The filtrate is recovered and if desired desalted using various chromatography media such as reverse phase and ion exchange media. The sample is then applied to a MALDI/TOF target or HPLC column and analyzed in a mass spectrometer the presence, absence or variation of the biomarkers. Higher recovery and concentration of these biomarkers is achieved with the present invention.

The present invention relates to sample preparation processes and kits for biological and bodily fluids for protenomic applications. More particularly, it relates to sample preparation processes and kits of biological and bodily fluids for the separation, recovery and identification of peptides and other biomarkers.

### BACKGROUND OF THE INVENTION

Recent studies have indicated that certain small weight proteins such as peptides, low molecular weight compounds and the like may be the indicators or biomarkers for certain illnesses and pathological states such as cancers, AIDS, diabetes and cardiovascular and neurological diseases. For example, in the article “Use of Proteomic Patterns in Serum to Identify Ovarian Cancer”, Petricoin, et. al., Lancet 359:572-577 (2002) the use of peptide patterns and deviations from them was indicated to be a potential method for screening ovarian cancer. In “Putative Pancreatic Cancer-Associated Diabetogenic Factor 2030 MW Peptide”, D. Basso et.al., Pancreas, vol. 24, No 1, pp8-14, a 2030 molecular weight peptide was indicated to be the biomarker for the presence of pancreatic cancer. In the April, 2002 volume of the Journal of the American Medical Association, the presence of two proteins, osteopontin and prostasin, were identified as indicators for the presence of ovarian cancer, see AMA, Osteopontin as a potential diagnostic biomarker for ovarian cancer . . . Kim J H; Skates S J; Mok Samuel et al; 287(3); April 2002.

Peptides and other such biomarkers are relatively small in size and present in relatively small amounts. Moreover, they are located in most biological materials such as blood, sera, plasma, spinal fluids, urine, cell lysates and the like.

These materials are relatively thick and viscous and contain a myriad of proteins and other materials that are difficult to isolate and identify and which, due to volume or size, mask the presence and detection of these biomarkers. Generally, less than one percent of the sample is made up of low molecular weight materials of interest.

WO 98/07036 teaches a methodology for recovering these peptides and using them to determine the health of an organism.

It teaches one to remove a large amount of bodily fluid (from 1 to 30 liters of hemodiafiltrate, up to 10 liters of an ascitic fluid or up to 50 liters of urine) which are then filtered through a 30 kiloDalton(kD) filter. This filtrate is treated by reducing its pH to between 2 and 4 and cooled to 4° C. The filtrate is then diluted with deionized water and its pH adjusted to 2.7. The diluted material is applied to a chromatographic column that binds the peptides.

Seven elution steps are then used to recover the peptides into seven samples. The seven samples are then each subjected to two or more chromatography steps. Aliquots of each eluant are then detected by mass spectrometry to form a peptide map. Once a map has been formed, individual targets can be identified and one can then use several additional chromatography steps to isolate the targets of interest and determine whether a variation exists in the peptides that would indicate a disease state.

As can be appreciated, there is the requirement for large volumes of starting material as well as extensive chromatography and dilution work to recover the biomarkers and then to isolate them. In addition, these steps are used to reduce the presence of inhibitory substances such as salts, protein fragments and the like that adversely affect proper analysis.

Many of the potential research and diagnostic applications of these biomarkers have not been explored because of the difficulty in their analysis and detection.

What is needed is a faster and more reliable system and process for isolating and identifying biomarkers for research and diagnostic use.

### SUMMARY OF THE INVENTION

The present invention provides a system, kit and process for the separation, recovery, purification and identification of biomarkers such as peptides and the like from biological and bodily fluids.

The process is to filter the sample through a centrifugal device containing an ultrafiltration membrane. The filtrate is recovered, and if desired, desalted using various chromatography media such as reverse phase media such as C18 or ion exchange media such as SCX media. The sample is then applied to an analytical device such as a mass spectrometer for detection and identification.

The system or kit of the present invention comprises all of the elements needed to run the process of the invention. It comprises a centrifugal device having an ultrafiltration membrane, chromatography media for desalting and purification and optionally, buffers, mass spectrometry matrix material and targets, and the like. The chromatography media is preferably bonded in a porous polymer scaffold or matrix, such as in a pipette tip, such as ZIPTIP® pipette tips.

It is an object of the present invention to provide a process for the sample preparation of biological and bodily fluids for protenomic applications comprising the steps of selecting a sample fluid containing one or more biomarkers, placing the fluid in a filtration device wherein the filter is an ultrafiltration membrane having a nominal molecular weight cutoff equal to or less than about 100 kiloDaltons (kD), applying a centrifugal force to the fluid in the device and recovering a filtrate, and applying mass spectrometry analysis to the recovered filtrate.

It is another object of the present invention to provide a process for the sample preparation of biological fluids for protenomic applications comprising the steps of selecting a biological fluid containing one or more biomarkers, placing the fluid in a centrifugal filtration device wherein the angle of the filter to the direction of force applied to the filter is from about −60 to +60 degrees from the force vector and the filter is an ultrafiltration membrane having a nominal molecular weight cutoff equal to or less than about 100 kiloDaltons (kDs), commonly less than about 50 kDs, preferably less than about 30 kDs, more preferably equal to or less than about 10 kDs, applying a centrifugal force to the fluid in the device and recovering a filtrate, and applying mass spectrometry analysis to the recovered filtrate.

It is a further object of the present invention to provide a kit for the isolation and purification of biomarkers comprising a filtration well device, wherein the filtration device has one or more wells, each well has one or more ultrafiltration membranes, said one or more membranes having a nominal molecular weight cutoff equal to or less than about 100 kiloDaltons (kD), one or more filtrate collection wells downstream of the one or more wells, and one or more portions of chromatography media contained in the one or more wells for the concentration and desalting of the filtrate. It is another object of the present invention to provide a kit for the isolation and purification of biomarkers comprising a filtration well device, wherein the filtration device has one or more wells, each well has one or more ultrafiltration membranes, said membrane(s) having a nominal molecular weight cutoff equal to or less than about 50 kiloDaltons (kD), one or more filtrate collection wells downstream of the one or more wells, and one or more portions of chromatography media for the desalting of the filtrate.

It is an additional object of the present invention to provide a kit for the isolation and purification of biomarkers comprising a filtration well device, wherein the filtration device has one or more wells, each well has one or more ultrafiltration membranes, said membrane having a nominal molecular weight cutoff equal to or less than about 30 kiloDaltons (kD), one or more filtrate collection wells downstream of the one or more wells, one or more portions of chromatography media for the desalting of the filtrate and optionally one or buffer solutions, mass spectrometry matrix and targets.

It is another object of the present invention to provide a kit for the isolation and purification of biomarkers comprising a filtration well device, wherein the filtration device has one or more wells, each well has one or more ultrafiltration membranes having a nominal molecular weight cutoff equal to or less than about 100 kiloDaltons (kD), one or more filtrate collection wells downstream of the one or more wells, one or more portions of chromatography media for the desalting of the filtrate wherein the chromatography media is fixed within a porous polymeric scaffolding or matrix contained within a housing such as a pipette tip.

### IN THE DRAIWNGS

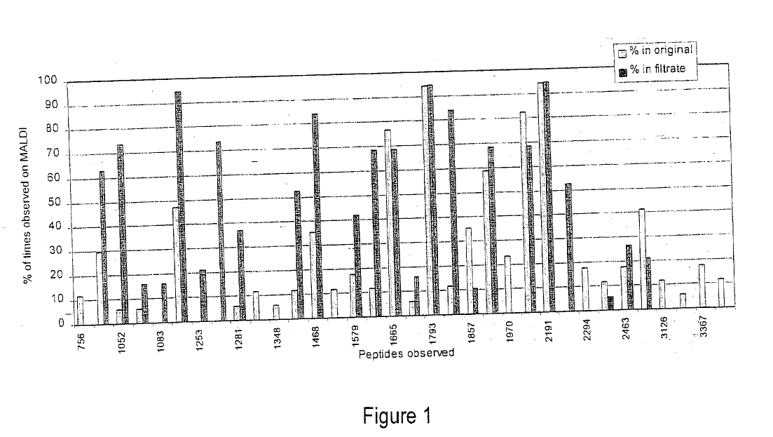

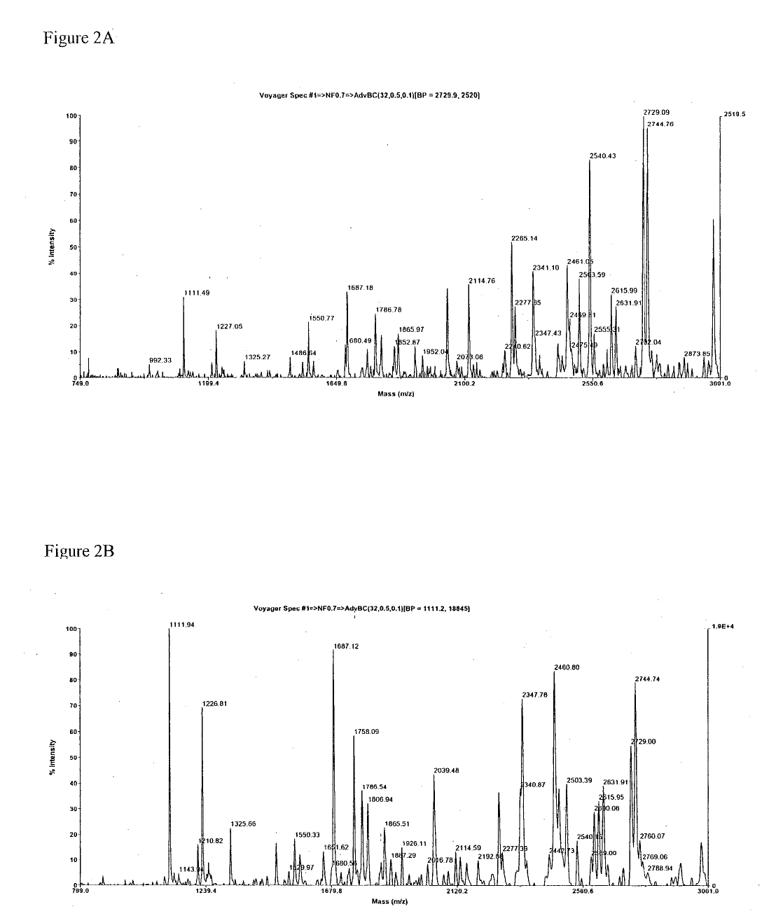

FIG. 1 shows a summary of the frequency of peptides observed before and after treatment of the bovine serum of Example 1 according to the present invention.

FIG. 2A shows MALDI spectra of Example 2 before treatment according to the present invention.

FIG. 2B shows MALDI spectra of Example 2 after treatment according to the present invention.

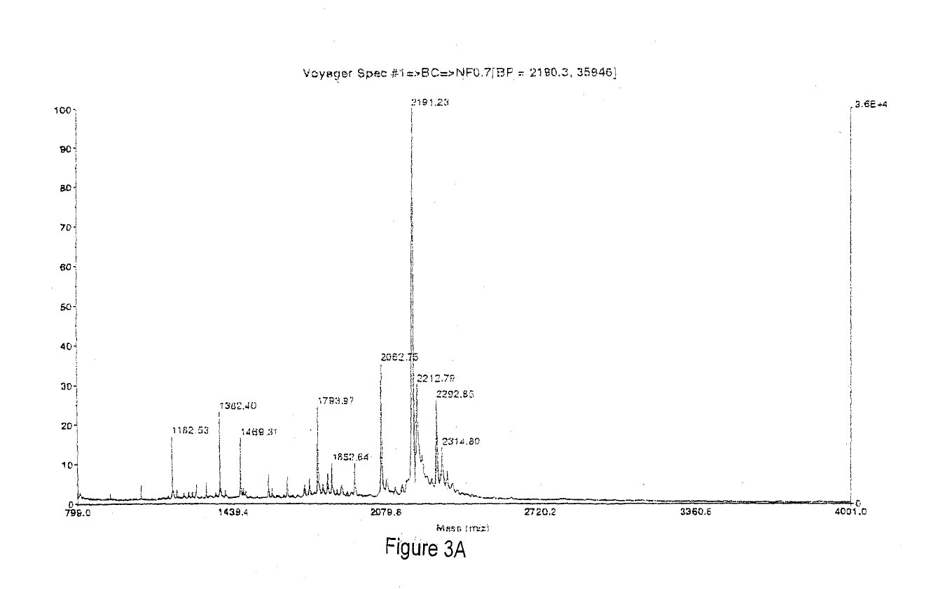

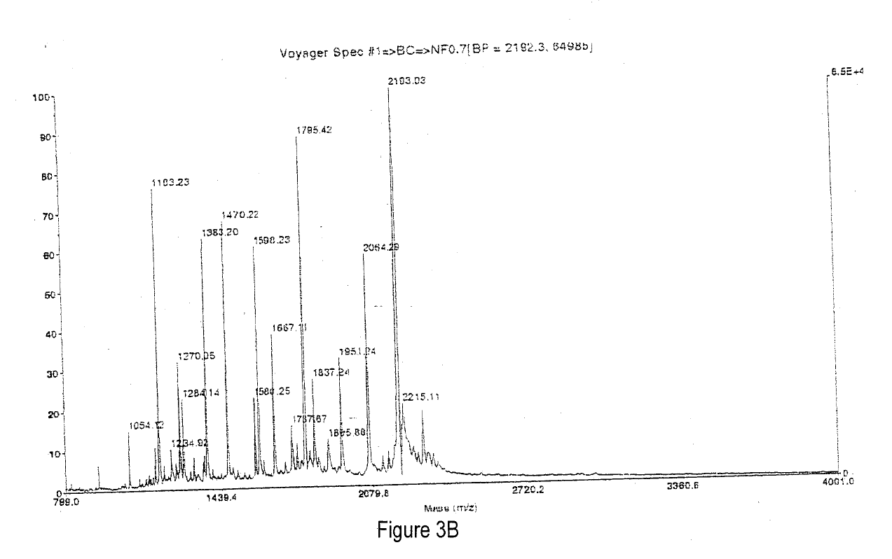

FIG. 3A shows MALDI spectra of Example 3 before treatment according to the present invention.

FIG. 3B shows MALDI spectra of Example 3 after treatment according to the present invention.

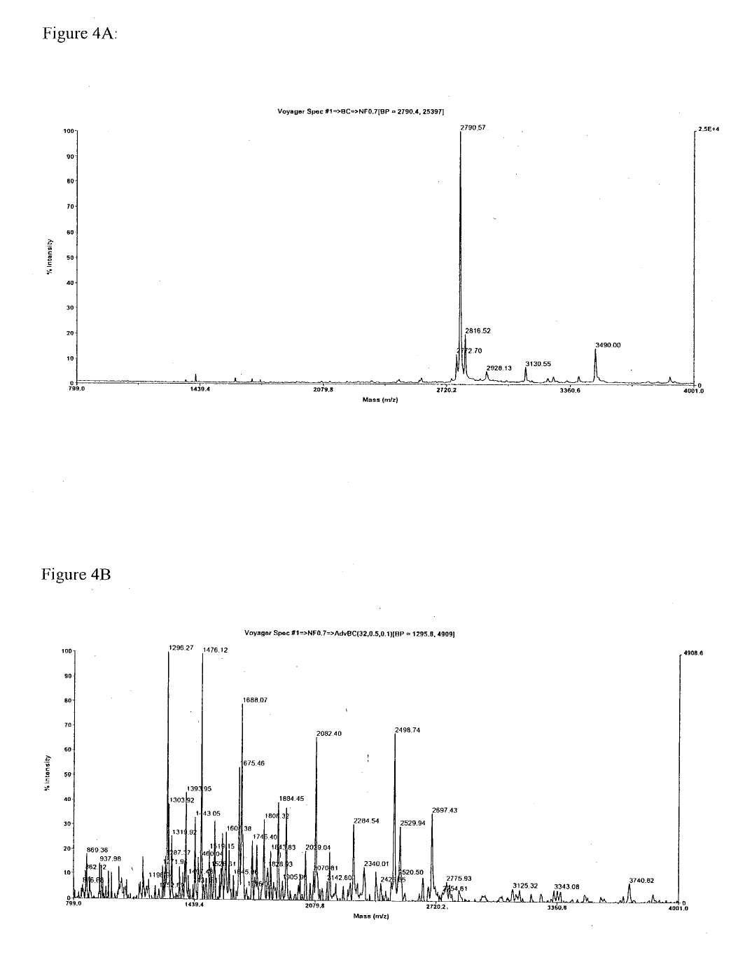

FIG. 4A shows MALDI spectra of Example 4 before treatment according to the present invention.

FIG. 4B shows MALDI spectra of Example 4 after treatment according to the present invention.

### DETAILED DESCRIPTION OF THE INVENTION

The present invention relates to a system, kit and process for the recovery and identification of biomarkers such as peptides and the like for the indicator or screen for various pathological states and conditions.

The process is to first obtain a sample to be tested. This may be obtained from a bodily fluid such as blood, sera, plasma, spinal fluids, synovial fluid, saliva, tears or ascites of a patient or test subject or a biological fluid such as a cell lysate or a cell culture or the like. The sample is filtered through an ultrafiltration (UF) membrane

The sample is filtered using a force such as centrifugation, positive pressure or negative pressure (vacuum) against the sample and the membrane to cause the material of a size at or below that of the filter pores to pass through the membrane and to be collected downstream in a filtrate collector or well. As the majority of available devices are designed for use with centrifugation, it is the currently preferred method for filtration with the present invention. It has been found that substantially all of the higher molecular weight materials can be removed quickly and effectively with this filtration step. Typically 95% to better than 99% of all higher molecular weight materials can be removed from a sample in a single filtration step. By quickly, it is meant that the average sample can be filtered in less than 30, preferably less than 20 minutes depending on the filter device and the viscosity of the fluid being treated.

The retentate, containing proteins and other materials too large to pass through the filter may be separately analyzed or thrown away.

Optionally, and preferably, the filtrate is then treated to remove any impurities such as salts, lipids and small molecules that would otherwise interfere with the identification of the biomarker. One could simply add the acidified filtrate to a vial containing a selected chromatography media such as a reverse phase media such as carbon 18 (C18) media or an ion exchange media such as SCX media and allow the two to be in contact for a time sufficient to bind the biomarkers. The remainder of the filtrate is then removed with a pipette or by decanting, the media is washed and an eluant such as an organic solvent for C18 media or a buffer at a different pH or salt concentration for other media is added to elute the biomarkers from the media.

Alternatively and preferably, the filtrate is treated using a device in which in the media is held in place or fixed in place such as is available in ZIPTIP® pipette tips available from Millipore Corporation of Billerica, Mass. and as taught in U.S. Pat. No. 6,200,474. This type of device has a housing with a three dimensional liquid permeable structure comprised of sorptive particles such as chromatography media entrapped in a porous polymeric matrix, preferably with the structure having an aspect ratio of less than about 10. Versions with different media including SCX and C18 are available. Other devices such as pipette tips or small centrifuge tubes and the like can use a frit, glass wadding, glue or other retaining structures at each end of the device with a column of media retained in between. These are equally acceptable for use in the present invention.

The pipette devices are preferred as they are typically made for treating small volumes of liquid with little or no loss of sample during the processing due to volume holdup or deadspace. One simply inserts the tip into the filtrate and repeatedly draws the fluid into and out of the device to ensure adequate capture of either the impurities or biomarkers. If the biomarkers are captured, then the tip is washed with deionized water or buffer to remove the unbound material and is placed into an elution bath and the elution material is moved repeatedly into and out of the device to recover the biomarkers. If the impurities are bound, one simply analyzes the remaining filtrate after this treatment.

The media filtrate containing the biomarkers is then applied to a MALDI/TOF, LC-MS or other mass spectrometry or other type of identification machine sampling device such as a HPLC column and analyzed for presence, absence or variation, and if present, for identification. If desired or required by the system used, one may use a mass spectrometry matrix material over the sample on the target such as CHCA (a-Cyano-4-hydroxycinnamic acid).

Devices suitable for use in this invention include but are not limited to a single well device having a horizontally oriented membrane (as to the direction of the filtration force) such as a CENTRICON® device available form Millipore Corporation of Billerica, Mass., a single well device having a vertical or substantially vertically oriented membrane such as a ULTRAFREE device available form Millipore Corporation of Billerica, Mass., a multiple membrane containing a single well device having a vertical or substantially vertically oriented membrane such as an AMICON® ULTRA™ device available from Millipore Corporation of Billerica, Mass. or a multiwell plate such as a MULTISCREEN® plate or an ULTRACELL™ plate available from Millipore Corporation of Billerica, Mass.

By vertical or substantially vertical, it is meant that the membrane length is oriented in a vertical direction or substantially parallel orientation as the force that is applied to it. In this way, the membrane is constantly swept by moving fluid that reduces or eliminates polarization or fouling the membrane allowing for faster and greater recovery of the filtrate. Typically, the membrane is parallel or 0 degrees to the direction of the force applied (such as the centrifugal force applied) although it may be at an angle to that force, typically from about +60 to about −60 degrees from the direction of the force applied, preferably from about +15 to about −15 degrees, more preferably from about about +12 to about −12 degrees and most preferably from about +6 to about −6 degrees to that force.

Also one can use a horizontal or substantially horizontally arranged membrane device, such as a TFF cassette including but limited to a PELLICON® XL cassette used in conjunction with a LABSCALE® TFF filtration system, both available from Millipore Corporation of Billerica, Mass. However, as such devices are relatively capital intensive and require larger volumes of fluid to process, they would be less preferred especially when dealing with smaller volumes of fluids as is common with laboratory or diagnostic work. Smaller TFF cassettes or devices may be available and to the extent that they are or become available, their use in the present invention is contemplated.

Suitable ultrafiltration membranes which can be utilized in the filtration device include those formed from regenerated cellulose, polyethersulfones, polysulphones and their copolymers, polyarylsulphones, polyimides, polyamides, polyvinylidene difluoride (PVDF) or the like. They may be formed as unsupported membranes or they may be formed as composite membranes having a support such as a microporous membrane or nonwoven support layer onto which the UF membranes are cast. Membranes with low protein binding are preferred to enhance the recovery of the biomarkers. UF membranes are well known and include ULTRACEL™ YM and PL cellulosic membranes available from Millipore Corporation of Billerica, Mass.

The nominal molecular weight cutoff of the selected filter should be 100 kD or less. Preferably it is about 50 kD or less, more preferably about 30 kDs or less and even down to about 10 kDs or less.

### EXAMPLE 1

Adult bovine serum was filtered in an Amicon®) Ultra™ device containing a UF membrane (Ultracel™ PL membrane) having a 10 kD NMWL available from Millipore Corporation of Billerica, Mass. in a centrifuge at 3000×g for 20 minutes. The ultrafiltrate was then desalted and concentrated in a ZIPTIP® pipette tip containing C18 media available from Millipore Corporation of Billerica, Mass.

The sample was then placed on a MALDI target covered by CHCA Matrix and analyzed for the presence of various peptides (total number of samples analyzed 19).

As a control, unfiltered adult bovine serum of the same batch was diluted in deionized water, desalted and concentrated in a ZIPTIP® pipette tip containing C18 media available from Millipore Corporation of Billerica, Mass. and placed on a MALDI target covered by CHCA Matrix and analyzed for peptides (total number of samples analyzed 17).

FIG. 1 shows a summary table of the frequency of peptides observed before and after treatment according to Example 1. As can be seen, the treatment provides one with a higher resolution of biomarkers, in this instance peptides, that is obtainable with the present invention as compared to the standard techniques.

### EXAMPLE 2

A sample of human serum was treated as in Example 1, with FIG. 2A showing the spectra obtained from the unfiltered, desalted filtrate and FIG. 2B showing the spectra obtained from ultrafiltered and desalted filtrate.

### EXAMPLE 3

A sample of adult bovine serum was treated as in Example 1, with FIG. 3A showing the spectra obtained from the unfiltered, desalted filtrate and FIG. 3B showing the spectra obtained from ultrafiltered and desalted filtrate.

### EXAMPLE 4

A sample of mouse serum was treated as in Example 1, with FIG. 4A showing the spectra obtained from the unfiltered, desalted filtrate and FIG. 4B showing the spectra obtained from ultrafiltered and desalted filtrate.

Examples 2-4 show the advantages and higher resolution obtained in ultrafiltering and then cleaning the filtrate sample before analysis.

The present invention provides a quick, simple methodology for isolating, concentrating and purifying biomarkers and a kit for doing so. It provides a relatively pure sample of low molecular constituents using small starting volumes in one or at most two steps in often under an hour's time. The present invention provides one with a fast, reliable and inexpensive way to find biomarkers and to use them as indicators of the state of health of an organism both in the laboratory and in the clinical or diagnostic setting.

What we claim:

1) A process for the sample preparation of biological fluids for proteomic applications comprising the steps of selecting a biological fluid, placing the fluid in an ultrafiltration membrane containing filtration device wherein the filter is an ultrafiltration membrane having a nominal molecular weight cutoff equal to or less than about 100 kiloDaltons (kD), applying a filtration force to the fluid in the device to create a filtrate that is essentially free of high molecular weight constituents and recovering the filtrate, and applying mass spectrometry analysis to the recovered filtrate.

2) A process for the sample preparation of biological fluids for proteomic applications comprising the steps of selecting a biological fluid, placing the fluid in an ultrafiltration membrane containing filtration device wherein the major plane of the filter to the direction of force applied to the filter is from about −60 to +60 degrees from the direction of the force vector and the filter is an ultrafiltration membrane having a nominal molecular weight cutoff equal to or less than about 100 kiloDaltons(kD), applying a filtration force to the fluid in the device to create a filtrate that is essentially free of high molecular weight constituents and recovering the filtrate, concentrating and desalting the filtrate and applying mass spectrometry analysis to the recovered filtrate.

3) The process of claim 1 wherein the filter is perpendicular to the direction of force applied to the filter.

4) The process of claim 1 wherein the filter has a cutoff of 50 kD or less.

5) The process of claim 1 wherein the filter has a cutoff of 30 kD or less.

6) The process of claim 1 wherein the filter has a cutoff of 10 kD or less.

7) The process of claim 1 wherein the filter is a regenerated cellulose.

8) The process of claim 1 wherein the filter has a cutoff of 10 kD or less and wherein the filter is a regenerated cellulose.

9) The process of claim 1 further comprising the step of desalting the filtrate before applying mass spectrometry by exposing the filtrate to a chromatography media.

10) The process of claim 1 further comprising the step of desalting the filtrate before applying mass spectrometry by exposing the filtrate to a chromatography media selected from the group consisting of reverse phase and ion exchange media.

11) The process of claim 1 further comprising the step of desalting the filtrate before applying mass spectrometry by exposing the filtrate to a chromatography media contained within a self-supportive resin matrix within a pipette tip.

12) The process of claim 1 wherein the biomarkers are selected from the group consisting of peptides and low molecular weight compounds.

13) The process of claim 1 wherein the biomarkers are obtained from a body fluid selected from the group consisting of blood, sera, plasma, spinal fluids, urine, synovial fluid, saliva, tears and ascites.

14) The process of claim 1 wherein the biomarkers are obtained from a biological fluid selected from the group consisting of cell culture media and cell lysates.

15) A kit for the isolation and purification of biomarkers comprising a filtration well device, wherein the filtration device has one or more wells, each well has one or more ultrafiltration membranes having a nominal molecular weight cutoff equal to or less than about 100 kiloDaltons (kD), one or more filtrate collection wells downstream of the one or more wells, and one or more portions of chromatography media for the desalting of the filtrate.

16) The kit of claim 15 wherein the filter has a cutoff of 50 kD or less.

17) The kit of claim 15 wherein the filter has a cutoff of 30 kD or less.

18) The kit of claim 15 wherein the filter has a cutoff of 10 kD or less.

19) The kit of claim 15 wherein the filter is regenerated cellulose.

20) The kit of claim 15 wherein the filter has a cutoff of 10 kD or less and wherein the filter is regenerated cellulose.

21) The kit of claim 15 wherein the chromatography media is selected from the group consisting of reverse phase and ion exchange media.

22) The kit of claim 15 wherein the chromatography media is selected from the group consisting of reverse phase and ion exchange media contained within a self-supportive resin matrix within a pipette tip.

23) The kit of claim 15 wherein the biomarkers are selected from the group consisting of peptides and low molecular weight compounds.

24) The kit of claim 15 further comprising one or buffer solutions.

25) The kit of claim 15 further comprising a mass spectrometry target.

26) The kit of claim 15 further comprising a mass spectrometry resin matrix.
